(19) United States
(12) Patent Application Publication
(10) Pub. No.: US 2005/0239889 A1
(43) Pub. Date: Oct. 27, 2005

(54) In vivo release of endogenous anti-microbial mediators by leukotriene B4 (LTB4) administration

(76) Inventors: Jean Gosselin, Sainte-Foy (CA); Louis Flamand, Sainte-Foy (CA); Pierre Borgeat, Sillery (CA)

Correspondence Address: KLAUBER & JACKSON, 411 HACKENSACK AVENUE, HACKENSACK, NJ, 07601, US

(21) Appl. No.: 11/114,899

(22) Filed: Apr. 26, 2005

Related U.S. Application Data
(60) Provisional application No. 60/564,947, filed on Apr. 26, 2004.

(51) Int. Cl. A61K 31/202

(52) U.S. Cl. 514/560

(57) ABSTRACT

The invention relates to the in vivo release of endogenous anti-microbial mediators using leukotriene B4 administration. The present invention furthermore relates to the use of leukotriene B4 for the treatment and/or prophylaxis of diseases that are positively influenced by such anti-microbial mediators.

### CROSS-REFERENCE TO RELATED APPLICATIONS

The present application is a non-provisional application claiming the priority of copending provisional application, U.S. Ser. No. 60/564,947, filed on Apr. 26, 2004, the disclosure of which is hereby incorporated by reference in its entirety. Applicants claim the benefits of this application under 35 U.S.C. § 119(e).

### BACKGROUND OF THE INVENTION

(a) Field of the Invention

The invention relates to the in vivo release of endogenous anti-microbial mediators, such as α-defensins and MIP-1β, using leukotriene B4 administration.

(b) Description of Prior Art

α-Defensins are small 3-4 kDa cationic antimicrobial peptides (29-34 amino acids) with three intramolecular disulfide bonds. α-Defensins are stored within cytoplasmic granules of neutrophils and are released in the extracellular milieu upon appropriate stimulation through a process referred to as degranulation. Neutrophil granules are defined as primary granules, which house α-Defensins, and secondary granules whose contents include lysosomal enzymes. α-Defensins are the most abundantly expressed neutrophilic proteins making up to 5% of the total cellular protein content. Considering the potent antimicrobial activity of the peptides and proteins making up these granules, the ability of neutrophils to release the content of these granules is essential in the fight against microbes. α-Defensins' antibacterial and anti-viral activities against enveloped viruses (including HIV), have been recognized for years. In most instances, damages to the bacterial cell membrane and viral envelope appear responsible for the antimicrobial activities of α-Defensins. In addition to α-Defensins, neutrophils are known to secrete various other immunomodulatory cytokines, including other anti-HIV mediators such as macrophage inflammatory protein 1 beta (MIP-1β).

It would be highly desirable to be provided with in vivo release of endogenous anti-microbial mediators, such as α-defensins and MIP-1β, using leukotriene B₄ administration.

### SUMMARY OF THE INVENTION

One aim of the present invention is to provide in vivo release of endogenous anti-microbial mediators, such as α-defensins and MIP-1β, using leukotriene B4 administration. The in vivo release in humans of beneficial anti-microbial mediators, such as α-defensins and MIP-1β, using leukotriene B₄ administration provides a way of demonstrating the beneficial effects of leukotriene B₄ administration. This is much closer to clinical practice than demonstrating it in in vitro cellular systems or in animal models. Many animals, such as mice, do not possess the ability of producing α-defensin or MIP-1β.

In accordance with the present invention there is provided a method for the in vivo release of an endogenous anti-microbial mediator in a human or animal comprising administering to a human or animal in need of such treatment, a pharmacologically acceptable therapeutically effective amount of exogenous LTB₄ agent.

The preferred mediator is an (α-defensin or MIP-1β.

The preferred LTB₄ agent is selected from the group consisting of:
leukotriene B₄ [5S,12R-dihydroxy-6,8,10,14(Z,E,E,Z)-eicosatetraenoic acid]; LTB4, 14,15-dihydro-LTB4 (“LTB3”), 17,18-dehydro-LTB4 (“LTB5”), 19-hydroxy-LTB4, 20-hydroxy-LTB4, and 5(S)hydroperoxy and 5-deoxy analogs thereof; 5-keto, 5(R)-hydroxy and 5(R)-hydroperoxy analogs of said LTB4 agent; leukotriene A4 (“LTA4”), 14,15-dihydro-LTA4 (“LTA3”), and 17,18-dehydro-LTA4 (“LTA5”);
14,15-dihydro-LTA4 methyl ester and LTB4 methyl ester; 12(R)-hydroxy-5,8,10,14(Z,Z,E,Z)-eicosatetraenoic acid (“12-HETE”), 5,6-dihydro-12-HETE, 14,15-dihydro-12-HETE,17,18-dehydro-12-HETE and 12(R)-hydroperoxy analogs thereof;
12-keto, 12(S)-hydroxy and 12(S)-hydroperoxy analogs of said LTB₄ agent; 5(S)-hydroxy-6,8,11,14(E,Z,Z,Z)-eicosatetraenoic acid (“5-HETE”), 14,15-dihydro-5-HETE, 17,18-dehydro-5-HETE, and 5(R)-hydroxy, 5-keto, 5(S)-hydroperoxy, 5(R)-hydroperoxy analogs thereof;
12-oxo-5,8,10(Z,Z,E)-dodecatrienoic acid, 15(S)-hydroxy-5,8,11,13(Z,Z,Z,E)-eicosatetraenoic acid (“15-HETE”), 5,6-dihydro-15-HETE, 17,18-dehydro-15-HETE and 15(R)-hydroxy, 15-keto, 15(S)-hydroperoxy, and 15(R)-hydroperoxy analogs thereof; 12(S)-hydroxy-5,8,10(Z,E,E)-heptadecatrienoic acid;
leukotrienes C₄, D₄ and E₄ and 14,15-dihydro or 17,18-dehydro analogs thereof; N-acyl or N-alkyl derivatives of leukotrienes C₄, D₄ and E4, and 14,15-dihydro or 17,18-dehydro analogs thereof;
5,12-dihydroxy-6,8,10,14-eicosatetraenoic acid, isomers thereof and 14,15-dihydro or 17,18-dehydro analogs thereof; 5,6-dihydroxy-7,9,11,14-eicosatetraenoic acid, isomers thereof and 14,15-dihydro or 17,18-dehydro analogs thereof; 5,15-dihydroxy-6,8,11,13-eicosatetraenoic acid, isomers thereof and 17,18-dehydro analogs thereof; 8-hydroxy-11(12)-epoxy-5,9,14-eicosatrienoic acid, hepoxilin A_{3,} isomers thereof and 5,6-dihydro or 14,15-dihydro or 17,18-dehydro analogs thereof; 10-hydroxy-11(12)-epoxy-5,8,14-eicosatrienoic acid, hepoxilin B₃, isomers thereof and 5,6-dihydro or 14,15-dihydro or 17,18-dehydro analogs thereof; 8,11,12-trihydroxy-5,9,14-eicosatrienoic acid, trioxilin A3, isomers thereof and 5,6-dihydro or 14,15-dihydro or 17,18-dehydro analogs thereof; 10,11,12-trihydroxy-5,8,14-eicosatrienoic acid, trioxilin B3, isomers thereof and 5,6-dihydro or 14,15-dihydro or 17,18-dehydro analogs thereof;
5(S),15(S)-dihydroxy-6,8,11,13(E,Z,Z,E)-eicosatetraenoic acid; 11(12)-epoxy-5,7,9,14-eicosatetraenoic acid, isomers thereof and 14,15-dihydro or 17,18-dehydro analogs thereof; 11,12-dihydroxy-5,7,9,14-eicosatetraenoic acid, isomers thereof and 14,15-dihydro or 17,18-dehydro analogs thereof; 8(9)-epoxy-5,10,12,14-eicosatetraenoic acid, isomers thereof and 5,6-dihydro or 17,18-dehydro analogs thereof; 8,9-dihydroxy-5,10,12,14-eicosatetraenoic acid, isomers thereof and 5,6-dihydro or 17,18-dehydro analogs thereof; 8,15-dihydroxy-5,9,11,13-eicosatetraenoic acid, isomers thereof and 5,6-dihydro or 17,18-dehydro analogs thereof; 14(15)-epoxy-5,8,10,12-eicosatetraenoic acid, isomers thereof and 5,6-dihydro or 17,18-dehydro analogs thereof; 14,15-dihydroxy-5,8,10,12-eicosatetraenoic acid, isomers thereof and 5,6-dihydro or 17,18-dehydro analogs thereof;
5-hydroxy-14(15)-epoxy-6,8,10,12-eicosatetraenoic acid, isomers thereof and 17,18-dehydro analogs thereof; 5,14,15-trihydroxy-6,8,10,12-eicosatetraenoic acid, lipoxin B₄, isomers thereof and 17,18-dehydro analogs thereof; 5,6,15-trihydroxy-7,9,11,13-eicosatetraenoic acid, lipoxin A₄, isomers thereof and 17,18-dehydro analogs thereof; 5(6)-epoxy-15-hydroxy-7,9,11,13-eicosatetraenoic acid, isomers thereof and 17,18-dehydro analogs thereof; 5-hydroxy-6,8,11,14-eicosatetraenoic acid, isomers thereof and 14,15-dihydro or 17,18-dehydro analogs thereof; 9-hydroxy-5,7,11,14-eicosatetraenoic acid, isomers thereof and 14,15-dihydro or 17,18-dehydro analogs thereof; 11-hydroxy-5,8,12,14-eicosatetraenoic acid, isomers thereof and 5,6-dihydro or 17,18-dehydro analogs thereof; 12-hydroxy-5,8,10,14-eicosatetraenoic acid, isomers thereof and 5,6-dihydro or 14,15-dihydro or 17,18-dehydro analogs thereof; 15-hydroxy-5,8,11,13-eicosatetraenoic acid, isomers thereof and 5,6-dihydro or 17,18-dehydro analogs thereof; 9-hydroxy-10,12-octadecadienoic acid and isomers thereof; 13-hydroxy-9,11-octadecadienoic acid and isomers thereof; 12(R)-hydroxy-5,8,14(Z,Z,Z)-eicosatrienoic acid and isomers thereof; 5(6)oxido- or 5,6-dihydroxy-8,11,14-eicosatrienoic acid, isomers thereof and 14,15-dihydro or 17,18-dehydro analogs thereof; 8(9)-oxido- or 8,9-dihydroxy-5,11,14-eicosatrienoic acid, isomers thereof and 5,6-dihydro or 14,15-dihydro or 17,18-dehydro analogs thereof; 11(12)-oxido- or 11,12-dihydroxy-5,8,14-eicosatrienoic acid, isomers thereof and 5,6-dihydro or 14,15-dihydro or 17,18-dehydro analogs thereof; 14(15)-oxido- or 14,15-dihydroxy-5,8,11-eicosatrienoic acid, isomers thereof and 5,6-dihydro or 17,18-dehydro analogs thereof;
8-hydroxy-5,9,11,14-eicosatetraenoic acid, isomers thereof and 5,6-dihydro or 14,15-dihydro or 17,18-dehydro analogs thereof;
20,20,20-trifluoromethyl-LTB₄; 19-methyl-LTB₄, 19,19-dimethyl-LTB₄, 19-fluoro-LTB₄, 19,19-difluoro-LTB₄, 18,20-difluro-LTB₄, 20-fluoro-LTB₄; 3-thio-LTB₄, 3-hydroxy-LTB_{4;}
3-methyl-LTB₄, 3,3-dimethyl-LTB₄, 3-fluoro-LTB₄, 3,3-difluoro-LTB₄, 2,3-difluoro-LTB_{4;}
LTB₄ methylsulfonylamide, LTB₄ methylamide, 1-tetrazole LTB_{4;}
LTB₄ agent is a salt thereof, an ester derivative thereof, and an ether derivative thereof.

In accordance with the present invention there is provided a method for the treatment or prophylaxis of a microbial infection, such as HIV or anthrax, in humans and animals by administering an LTB₄ agent.

The administration is effected orally, intraarterially, intravenously, intraperitoneally, subcutaneously, intramuscularly, sublingually, intranasally, parenterally, topically, by inhalation or by suppository.

All publications, patents and patent applications are herein incorporated by reference in their entirety to the same extent as if each individual publication, patent or patent application was specifically and individually indicated to be incorporated by reference in its entirety.

### BRIEF DESCRIPTION OF THE DRAWINGS

FIG. 1 illustrates dose-dependent release of α-Defensins following intravenous administration of LTB₄ to human subjects;

FIG. 2 illustrates release of MIP-β following intravenous administration of LTB₄ to human subjects; and
FIG. 3 illustrates release of α-Defensins following subcutaneous administration of LTB4 to monkeys.

### DETAILED DESCRIPTION OF THE INVENTION

The present invention pertains to the use of leukotriene B4 (LTB₄) as a method to induce, in humans and animals, the release of antimicrobial agents such as but not restricted to α-Defensins and MIP-1β.

The induction of the release of antimicrobial agents such as α-defensins and MIP-1βby the administration of LTB₄ agents is beneficial to any disease where the antimicrobial agents, in particular α-defensins and MIP-1β, are known to have a beneficial effect. Such diseases include HIV and anthrax. It has recently been found that α-defensins inhibit lethal factor (LF) produced by bacillus anthracis. LF plays a major role in anthrax pathogenesis.

The present invention hence also includes a method for the treatment or prophylaxis of a microbial infection, such as HIV or anthrax, by the administration of LTB₄ agents.

The leukotriene B₄ (LTB₄) agent of the present invention is either LTB₄ or certain structurally related polyunsaturated fatty acids, or substances structurally unrelated to fatty acids, which stimulate the synthesis of LTB₄ or other LTB₄ agents by cells, or mimic their biological activity. They are either natural substances or analogs of such natural substances. All of the LTB₄ agents can be obtained by chemical synthesis by methods described in the literature and most are commercially available.

As used herein, the term “LTB₄ agent” means one or more of the following polyunsaturated fatty acids, which in addition to LTB₄ itself, are analogs of LTB₄, or precursors or metabolites of LTB₄ or LTB₄ analogs: LTB₄, 14,15-dihydro-LTB₄, 17,18-dehydro-LTB₄, 19-hydroxy-LTB₄, 20-hydroxy-LTB₄ and their 5(R)-hydroxy, 5-keto, 5(S)hydroperoxy, 5(R)-hydroperoxy and 5-deoxy analogs; LTA₄;
14,15-dihydro-LTA₄, 17,18-dehydro-LTA₄; 5(S)-hydroxy-6,8,11,14(E,Z,Z,Z)-eicosatetraenoic acid (“5-HETE”), 14,15-dihydro-5-HETE, 17,18-dehydro-5-HETE, and their 5(R)-hydroxy, 5-keto, 5(S)-hydroperoxy, 5(R)-hydroperoxy analogs; 12(R)-hydroxy-5,8,10,14(Z,Z,E,Z)-eicosatetraenoic acid (“12-HETE”), 5,6-dihydro-12-HETE, 14,15-dihydro-12-HETE, 17,18-dehydro-12-HETE and their 12(S)-hydroxy, 12-keto, 12(S)-hydroperoxy and 12(R)-hydroperoxy analogs and 12-oxo-5,8,10(Z,Z,E)-dodecatrienoic acid, 15(S)-hydroxy-5,8,11,13(Z,Z,Z,E)-eicosatetraenoic acid (“15-HETE”), 5,6- dihydro-15-HETE, 17,18-dehydro-15-HETE and their 15(R)-hydroxy, 15-keto, 15(S)-hydroperoxy, and 15(R)-hydroperoxy analogs.

The term LTB4 agent also includes other derivatives of polyunsaturated fatty acids; some are derived from the cyclooxygenase pathways, the lipoxygenase pathways (5-, 12- and 15-lipoxygenases) or the cytochrome P450 pathways; others are isomers, analogs or derivatives of naturally formed compounds: 12(S)-hydroxy-5,8,10(Z,E,E)-heptadecatrienoic acid; leukotrienes C₄, D₄ and E₄ and their 14,15-dihydro or 17,18-dehydro analogs; N-acyl or N-alkyl derivatives of leukotrienes C₄, D₄ and E₄, and their 14,15-dihydro or 17,18-dehydro analogs; all isomeric 5,12-dihydroxy-6,8,10,14-eicosatetraenoic acids and their 14,15-dihydro or 17,18-dehydro analogs; all isomeric 5,6-dihydroxy-7,9,11,14-eicosatetraenoic acids and their 14,15-dihydro or 17,18-dehydro analogs; all isomeric 5,15-dihydroxy-6,8,11,13-eicosatetraenoic acids (including 5(S),15(S)-dihydroxy-6,8,11,13(E,Z,Z,E)-eicosatetraenoic acid) and their 17,18-dehydro analogs; all isomeric 8-hydroxy-11(12)-epoxy-5,9,14-eicosatrienoic acids (including hepoxilin A₃) and their 5,6-dihydro or 14,15-dihydro or 17,18-dehydro analogs; all isomeric 10-hydroxy-11(12)-epoxy-5,8,14-eicosatrienoic acids (including hepoxilin B₃) and their 5,6-dihydro or 14,15-dihydro or 17,18-dehydro analogs; all isomeric 8,11,12-trihydroxy-5,9,14-eicosatrienoic acids (including trioxilin A₃) and their 5,6-dihydro or 14,15-dihydro or 17,18-dehydro analogs; all isomeric 10,11,12-trihydroxy-5,8,14-eicosatrienoic acids (including trioxilin B₃) and their 5,6-dihydro or 14,15-dihydro or 17,18-dehydro analogs; all isomeric 11(12)-epoxy-5,7,9,14-eicosatetraenoic acids and their 14,15-dihydro or 17,18-dehydro analogs; all isomeric 11,12-dihydroxy-5,7,9,14-eicosatetraenoic acids and their 14,15-dihydro or 17,18-dehydro analogs; all isomeric 8(9)-epoxy-5,10,12,14-eicosatetraenoic acids and their 5,6-dihydro or 17,18-dehydro analogs; all isomeric 8,9-dihydroxy-5,10,12,14-eicosatetraenoic acids and their 5,6-dihydro or 17,18-dehydro analogs; all isomeric 8,15-dihydroxy-5,9,11,13-eicosatetraenoic acids and their 5,6-dihydro or 17,18-dehydro analogs; all isomeric 14(15)-epoxy-5,8,10,12-eicosatetraenoic acids and their 5,6-dihydro or 17,18-dehydro analogs; all isomeric 14,15-dihydroxy-5,8,10,12-eicosatetraenoic acids and their 5,6-dihydro or 17,18-dehydro analogs; all isomeric 5-hydroxy-14(15)-epoxy-6,8,10,12-eicosatetraenoic acids and their 17,18-dehydro analogs; all isomeric 5,14,15-trihydroxy-6,8,10,12-eicosatetraenoic acids (including lipoxin B₄) and their 17,18-dehydro analogs; all isomeric 5,6,15-trihydroxy-7,9,11,13-eicosatetraenoic acids (including lipoxin A₄) and their 17,18-dehydro analogs; all isomeric 5(6)-epoxy-15-hydroxy-7,9,11,13-eicosatetraenoic acids and their 17,18-dehydro analogs; all isomeric 5-hydroxy-6,8,11,14-eicosatetraenoic acids and their 14,15-dihydro or 17,18-dehydro analogs; all isomeric 8-hydroxy-5,9,11,14-eicosatetraenoic acids and their 5,6-dihydro or 14,15-dihydro or 17,18-dehydro analogs; all isomeric 9-hydroxy-5,7,11,14-eicosatetraenoic acids and their 14,15-dihydro or 17,18-dehydro analogs; all isomeric 11-hydroxy-5,8,12,14-eicosatetraenoic acids and their 5,6-dihydro or 17,18-dehydro analogs; all isomeric 12-hydroxy-5,8,10,14-eicosatetraenoic acids and their 5,6-dihydro or 14,15-dihydro or 17,18-dehydro analogs; all isomeric 15-hydroxy-5,8,11,13-eicosatetraenoic acid and their 5,6-dihydro or 17,18-dehydro analogs; all isomeric 9-hydroxy-10,12-octadecadienoic acids; all isomeric 13-hydroxy-9,11-octadecadienoic acids; 12(R)-hydroxy-5,8,14(Z,Z,Z)-eicosatrienoic acid; all isomeric 5(6)oxido- or 5,6-dihydroxy-8,11,14-eicosatrienoic acids and their 14,15-dihydro or 17,18-dehydro analogs; all isomeric 8(9)-oxido- or 8,9-dihydroxy-5,11,14-eicosatrienoic acids and their 5,6-dihydro or 14,15-dihydro or 17,18-dehydro analogs; all isomeric 11(12)-oxido- or 11,12-dihydroxy-5,8,14-eicosatrienoic acids and their 5,6-dihydro or 14,15-dihydro or 17,18-dehydro analogs; all isomeric 14(15)-oxido- or 14,15-dihydroxy-5,8,11-eicosatrienoic acids and their 5,6-dihydro or 17,18-dehydro analogs.

The term LTB₄ also includes variants which are non-covalently modified fatty acids such as the sodium or the potassium salts of the LTB₄ agents.

The term LTB₄ agent also includes variants where a modification is introduced into the molecule by reacting targeted functional groups of the fatty acid with an organic derivatizing agent that is capable of reacting with the selected functional group (yielding for example, ester and ether derivatives of LTB₄ agent) or to cause intramolecular rearrangement (such as the formation of lactones with hydroxylated fatty acids).

The term “salt thereof” is intended to mean pharmaceutically acceptable base addition salts obtainable by treating the acid form of a functional group, such as a carboxylic acid, with appropriate bases such as inorganic bases, for example alkaline metal hydroxides; typically sodium or potassium hydroxide; alkaline metal carbonates; typically sodium or potassium carbonate or hydrogencarbonate; or ammonia; or organic bases, for example primary, secondary, or tertiary amines, alkaline metal or alkaline earth metal alcoholates, for example sodium methanolate, sodium ethanolate, or potassium ethanolate. Preferred salts are base addition salts with sodium or potassium hydroxide.

The term “ester derivatives” is intended to mean pharmaceutically acceptable esters obtainable by treating the acid or acid derivative form of a functional group with any typical esterification agent known to the person skilled in the art. Examples of esters are C₁₋₄ alkyl esters, such as methyl ester, ethyl ester, n-propyl ester, i-propyl ester, n-butyl ester, i-butyl ester, s-butyl ester, and t-butyl ester.

The term “ether derivatives” is intended to mean pharmaceutically acceptable ethers obtainable by treating the alcohol or alcohol derivative form of a functional group with any typical etherification agent known to the person skilled in the art. Examples of ethers are C₁₋₄ alkyl ethers, such as methyl ether, ethyl ether, n-propyl ether, i-propyl ether, n-butyl ether, i-butyl ether, s-butyl ether, and t-butyl ether.

The term “lactones” in the context of the present invention is easily understood by the person skilled in the art as an intramolecular ester formed in a molecule containing a hydroxy group and e.g. a carboxylic acid group. In e.g. the molecule LTB₄ there is a hydroxy group positioned in the δ-position relative to the carboxylic acid group, and this molecule is therefore capable of forming a δ-lactone.

The resulting compounds may have altered biological activity and/or bioavailability. Thus, the covalently modified fatty acid can be a pro-drug with reduced biological activity which upon in vivo administration is slowly transformed into a more active molecule (underivatized LTB₄ agent). Variants may also be metabolically stable and biologically active analogs of LTB₄ agents altered in a way that will result in retarded disposition of the compound (decreased metabolism and/or elimination). Variants with modifications at the omega end (such as 20,20,20-trifluoromethyl-LTB₄) show increased resistance to omega-oxidation (a catabolic process of unsaturated fatty acids); other variants with modification at the omega end at the level of carbons 13 to 20 (such as 19-methyl-LTB₄ or 19,19-dimethyl-LTB₄ or 19-fluoro-LTB₄ or 19,19-difluoro-LTB₄ or 18,20-difluro-LTB₄ or 20-fluoro-LTB₄) may show increased resistance to omega-oxidation and variants with modifications at the carboxylic end, at the level of carbon 1, 2, 3 or 4 (for example, 3-thio-LTB₄, 3-hydroxy-LTB₄, 3-methyl-LTB₄ or 3,3-dimethyl-LTB₄ or 3-fluoro-LTB₄ or 3,3-difluoro-LTB₄ or 2,3-difluoro-LTB₄, LTB₄ methylsulfonylamide, LTB₄ methylamide, 1-tetrazole LTB₄), may show increased metabolic resistance to beta-oxidation and/or to elimination (such as uptake by probenecide-sensitive organic acid transporter). Other variants with modification(s) at carbon 12, such as 12(R)-methyl-LTB₄, may show increased resistance to reduction of the 11,12 double bond (a metabolic pathway of LTB₄). Other variants are analogs of LTB₄ agents with structural changes, such as changes in chain length (chain length increased or decreased by up to 4 carbons), addition of double bond(s), saturation of double bond(s), changes in double bond(s) geometry (cis to trans or vice versa), change of double bond(s) for triple bond(s), change in the configuration of one or several functional group(s) (R to S or S to R), or where one or several functional group(s) or substituent(s) are either removed, added or changed for other functional groups or substituents (including but not limited to hydroperoxyl, carbonyl, sulfhydryl, sulfoxide, sulfone, cysteinyl, glutathionyl, cysteinyl-glycine, methyl, isopropyl, benzyl, chloro, fluoro), or where the positions of one or several functional groups and/or one or several double bonds has been moved by one, two or three carbons relative to the omega end. The LTB₄ agent may be a variant carrying one or several of the above mentioned structural modifications.

The LTB₄ agents and variants of LTB₄ agents are structurally related to LTB₄ and bind or may bind with different affinities to either the cell surface binding sites of LTB₄ (or other related eicosanoids, including but not limited to 5-HETE, LTD₄, lipoxin A₄) present on various leukocytes (and other cell types), or to the nuclear binding site of LTB₄, the transcription factor PPARo (peroxisome proliferator-activated receptor alpha) (Devchand P. R., et al., Nature 384:39, 1996), or to other unknown binding sites of LTB₄, resulting in the expression of the biological activities of LTB₄ and LTB₄ agents. The LTB₄ agents and variants show or may show biological activities qualitatively similar to that of LTB₄ (but may be more or less active than LTB₄ itself) and thus can be expected to exert an antiviral activity similar to that of LTB₄. The LTB₄ agents and variants thereof are included within the scope of this invention.

The term LTB₄ agent also includes agents not structurally related to LTB₄ including but not limited to the chemotactic peptide formyl-met-leu-phe (fMLP) (and analogs such as N-formyl-nle-leu-phe, N-formyl-met-leu-phe-benzylamide, N-formyl-met-leu-phe-methyl-ester and N-formyl-Nle-leu-phe-nle-tyr-lys), the complement fragment C5a and analogs, and the biologically active phospholipid platelet-activating factor, 1-0-hexadecyl-2-0-acetyl-sn-glycero-3-phospho-choline (and analogs such as 1-0-octadecyl-2-0-sn-glycero-3-phosphocholine and 1-0-hexadecyl-2-N-methyl-carbamyl-sn-glycero-3-phosphocholine) that stimulate or may stimulate the release of unsaturated fatty acids in cells (mainly arachidonic acid) and consequently the formation of one or several LTB₄ agents, and may therefore exhibit an antiviral activity similar to that of LTB₄. The above-mentioned LTB₄ agents not structurally related to LTB₄ are thus included within the scope of this invention.

The term LTB₄ agent also includes formulations of compounds which might contain a mixture of two or several LTB₄ agents or an LTB₄ agent and one or several equally or less active isomer(s) of the LTB₄ agent (positional, geometrical or optical isomers).

The term LTB₄ agent also includes antibodies to the LTB₄ receptor, or anti-idiotypic antibodies to antibodies raised against LTB₄ or one of the above-mentioned analogs or variants of LTB₄, which can be expected to elicit an LTB₄-like biological response, such as an antiviral effect.

The microbial infections which may be treated with the LTB₄ agent, in accordance with the invention, are infections caused by human and/or animal viruses, bacteria, fungus, protozoa among others, more specifically HIV and anthrax.

The expression “human and/or animal viruses” is intended to include, without limitation, DNA and RNA viruses in general and Retroviridae. DNA viruses include parvoviridae, papovaviridae, adenoviridae, herpesviridae, poxviridae and hepadnaviridae. RNA viruses include picornaviridae, togaviridae, orthomyxoviridae, paramyxoviridae, coronaviridae, reoviridae, oncornaviridae and filoviridae.

The therapeutically effective amount of the LTB₄ agent to be administered will vary with the particular LTB₄ agent used, the type or mode of administration, the concurrent use of other active compounds, host age and size, type, severity and spread of infection, response of individual patients, and the like. In the case of LTB₄, it will be administered in sufficient doses to obtain an effective peak or steady-state concentration of about 0.25 nM to 1000 nM, preferably of about 0.25 nM to 25 nM, and more preferably of about 0.25 nM to about 2.5 nM. An effective dose amount of the LTB₄ agent is thus be determined by the clinician after a consideration of all the above-mentioned criteria. In the case of LTB₄ agents other than LTB₄ which have a different biological activity, the effective peak or steady-state concentration required may be different, for instance up to 10 μM. The dosage amount of agent necessary to obtain the desired concentrations in blood can be determined by pharmacokinetic studies, as described in Marleau et al., J. Immunol. 150: 206, 1993, and Marleau et al, Br. J. Pharmacol. 112: 654, 1994.

Any suitable type or mode of administration may be employed for providing a mammal, especially a human with an effective dosage of a LTB₄ agent of the present invention. For example, intravenous, subcutaneous, inhalation, sublingual, intranasal, oral, parenteral and topical may be employed. Dosage forms include tablets, capsules, powders, solutions, dispersions, suspensions, creams, ointments and aerosols.

The pharmaceutical compositions of the present invention comprise a LTB₄ agent as an active ingredient, and a pharmaceutically acceptable carrier and optionally other therapeutic ingredients.

It should be recognized that the LTB₄ agent can be used in a variety of ways in vivo. It can be formulated into pharmaceutical compositions according to any known methods of preparing pharmaceutically useful compositions. In this manner, the fatty acid is combined in admixture with a pharmaceutically acceptable carrier vehicle. Suitable vehicles and their formulation, including human proteins, e.g., human serum albumin, are described for instance in Remington's Pharmaceutical Sciences (16th ed. Osol, A., ed., Mack, Easton, Pa. [1980]). In order to form a pharmaceutically acceptable composition suitable for effective administration, such compositions will contain a therapeutically effective amount of the LTB₄ agent or amount resulting in antiviral activity, together with a suitable amount of carrier vehicle. The amounts required for antiviral effects can be determined by in vivo pharmacological studies.

The LTB₄ agent can be formulated as a sterile pharmaceutical composition for therapeutic use which is suitable for intravenous or intraarterial administration. The product may be in a solvent-free form and ready to be reconstituted for use by the addition of a suitable carrier or diluent, or alternatively, it may be in the form of solution which may be aqueous or organic.

For reconstitution of a solvent-free product in accordance with the present invention, one may employ a sterile diluent, which may contain materials generally recognized for approximating physiological conditions. In this manner, the sterile diluent may contain salts and/or buffering agents to achieve a physiologically acceptable tonicity and pH, such as sodium chloride, phosphate and/or other substances which are physiologically acceptable and/or safe for use.

When used as an aqueous solution, the pharmaceutical composition will for the most part contain many of the same substances described above for the reconstitution of a solvent-free product. When used in solution in an organic solvent, a small volume of the solution containing the fatty acid will be diluted with an aqueous solution that will contain many of the same substances described above for the reconstitution of a solvent-free product. The pharmaceutical composition, for the most part, will thus contain many of the same substances described above for the reconstitution of a solvent-free product.

The LTB₄ agent useful in the methods of the present invention may be employed in such forms as, for example, sterile solutions for injection or encapsulated (for instance in liposomes) or embedded (for example in suppositories) for slower long-lasting release.

The LTB₄ agent may be used in combination with other agents including, but not limited to, anti-viral agents, anti-cancer agents, immunosuppressive agents, anti-inflammatory agents, cytokines, retinoids and compounds that may reduce uptake, elimination or metabolism of the LTB₄ agent such as probenecide, dipyridamole or clofibrate.

Where the subject LTB₄ agent is to be administered to a host as an anti-viral agent, the agent may be administered, for example, intraarterially, intravenously, intraperitoneally, subcutaneously, intramuscularly, by injection, by inhalation, by suppository, or the like. Because of the high cost of most LTB₄ agents and their chemical stability, injection of LTB₄ may represent the most advantageous form of administration of the composition of the present invention to a patient in order to achieve a better control of the dosage. The mode of administration by injection includes continuous infusion as well as single or multiple boluses. Given the short half-life of some LTB₄ agents in the circulation (Marleau et al., Br. J. Pharmacol. 112: 654, 1994), their administration as single or multiple boluses may imply the simultaneous use of agents to retard elimination of LTB₄ agent and/or to inhibit its metabolism, or alternatively, the use of analogs of LTB₄ agents with prolonged half-life in the circulation. Useful administration type or mode also includes the use of implantable internal pumps for continuous infusion into a blood vessel or at different sites such as the peritoneal cavity or subcutaneously. Such techniques are disclosed in Cecil's Text Book of Medicine (Chapter 164, 19th Edition, 1992) for the treatment of hepatic cancers. Transdermal administration by means of a patch containing the LTB₄ agent may also be a useful administration mode.

Additional pharmaceutical methods may be employed to control the duration of action. For example, controlled release preparations may be achieved through the use of macromolecules to complex or absorb the agent. The controlled delivery may be achieved by selecting appropriate macromolecules (for example, polyesters, polyamino acids, polyvinyl pyrrolidone, ethylene-vinyl acetate, methyl cellulose, carboxymethyl cellulose, protamine sulfate or serum albumin, the appropriate concentration of macromolecules, as well as the methods of incorporation. In this manner, release of the agent can be controlled.

Another possible method useful in controlling the duration of action by controlled release preparations is the incorporation of the agent into particles of a polymeric material such as polyesters, polyamino acids, hydrogels, poly(lactic acid), or ethylene-vinyl acetate copolymers.

Instead of incorporating the subject fatty acids into polymeric particles, it is also possible to entrap these materials in microcapsules prepared, for instance, by coacervation techniques or by interfacial polymerization (for example, hydroxymethyl cellulose or gelatin microcapsules and polymethyl methacrylate microcapsules, respectively), in colloidal drug delivery systems (for example, liposomes, albumin microspheres, microemulsions, nanoparticles and nanocapsules) or in macroemulsions. Such techniques are disclosed in Remington's Pharmaceutical Sciences (16th ed. Osol, A., ed., Mack, Easton, Pa. [1980]).

The compositions include compositions suitable for oral or parenteral administration. Conveniently they are presented in unit dosage form and prepared by any of the methods well-known in the art of pharmacy.

In practical use, the LTB₄ agent can be combined as the active ingredient in intimate admixture with a pharmaceutical carrier according to conventional pharmaceutical compounding techniques. The carrier may take a wide variety of forms depending on the form of preparation desired for administration. In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions; elixirs and solutions; or carriers such as starches, sugars, microcrystalline cellulose, diluents, granulating agents, lubricants, binders, disintegrating agents and the like in the case of oral solid preparations such as, for example, powders, capsules and tablets. If desired, tablets may be coated by standard aqueous or non-aqueous techniques.

Pharmaceutical compositions of the present invention suitable for oral administration may be presented as discrete units such as capsules, cachets or tablets each containing a predetermined amount of the LTB₄ agent, as a powder or granules or as a solution or suspension in an aqueous liquid, a non-aqueous liquid, an oil-in-water emulsion or a water-in-oil emulsion. Such compositions may be prepared by any of the methods of pharmacy such methods including the step of bringing the LTB₄ agent into association with the carrier which includes one or more necessary ingredients. In general, the compositions are prepared by uniformly and intimately admixing the LTB₄ agent with liquid carriers or finely divided solid carriers or both, and then, if necessary, shaping the product into the desired presentation. For example, a tablet may be prepared by compression of molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

It will be understood that the LTB₄ agent is to be administered in pharmacologically or physiologically acceptable amounts, by which is to be understood amounts not harmful to the patient, or amounts where any harmful side effects in individual patients are outweighed by the benefits. Similarly, the LTB₄ agent is to administered in a therapeutically effective amount, which is to be understood is an amount meeting the intended therapeutic objectives, and providing the benefits available from administration of LTB₄ agent.

The present invention will be more readily understood by referring to the following examples which are given to illustrate the invention rather than to limit its scope.

### EXAMPLE I

In Vivo Release of α-Defensins and MIP-1β Following LTB₄ Administration to Humans

Material and Methods

Plasma samples were obtained from 19 HIV-uninfected human volunteers taking part in a Phase I clinical study on the safety and tolerability of intravenously (i.v.) administered bolus of LTB₄. In brief, 3 individuals received saline (placebo) and 16 (4 subjects/group) were administered LTB₄ in doses ranging from 0.05 μg/kg to 50 μg/kg per day for 7 consecutive days. Venous blood was collected twice before injection (-5 and -2 minutes) to establish baseline values. Following the i.v. administration of saline or LTB₄, blood samples were drawn at 0.5, 1, 2, 4, 6, and 24 hours post-injection into blood collection tubes, using EDTA as anti-coagulant. Immediately after blood collection, the samples were put on ice until processed. All plasma isolation procedures were carried out at 4° C. Plasma samples were stored at −80° C. until assayed by ELISA for α-Defensins (HyCult Biotechnology bv, The Netherlands) and MIP-β(Amersham/Pharmacia, Baie d'Urfé, Canada).

Results

Release of α-Defensins and MIP-1β Following LTB₄ Administration to Humans

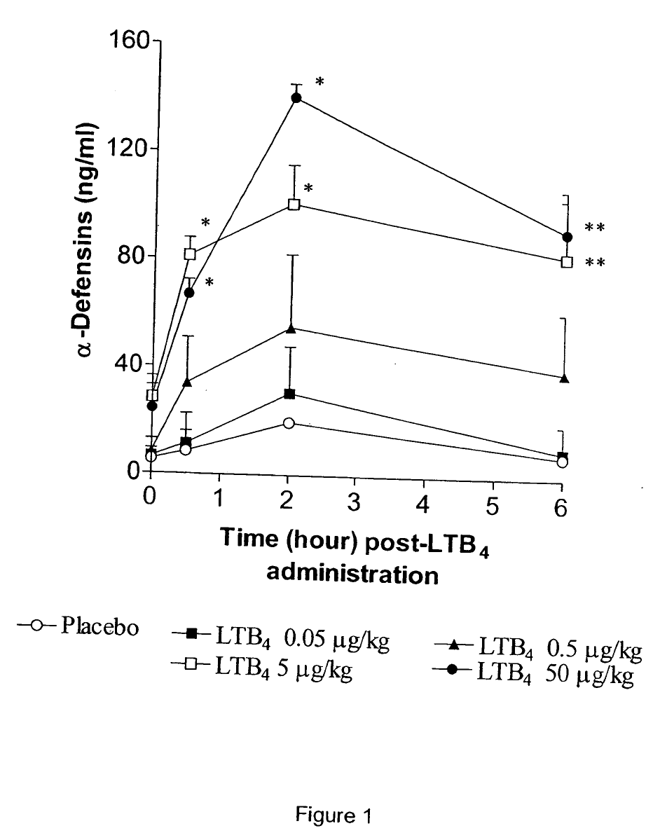

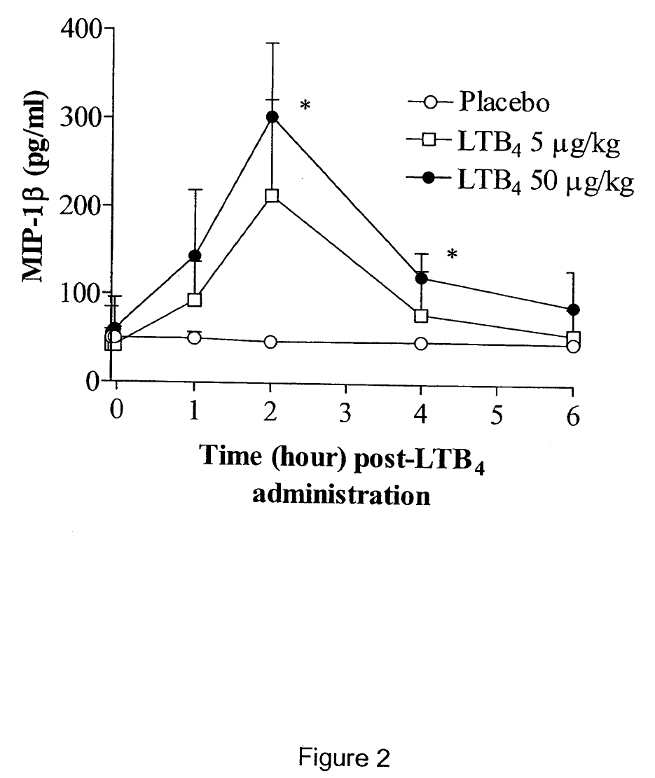

Healthy volunteers participating in a Phase I clinical study on the safety and tolerability of intravenously administered LTB₄, were randomized into 5 groups: placebo (n=3), LTB₄ 0.05 μg/kg (n=4), LTB₄ 0.5 μg/kg (n=4), LTB₄ 5 μg/kg (n=4) and LTB₄ 50 μg/kg (n=4). LTB₄ was administered once daily for 7 consecutive days and plasma samples (0-6 hours post-LTB₄ administration) from all 19 volunteers were tested for the presence of α-Defensins using a commercial ELISA kit. The mean ± standard error of α-Defensins releases (ng/ml plasma) for each group are presented in FIG. 1 with statistical analysis performed using the placebos as control group. Healthy individuals received a placebo (3 subjects) or doses of LTB₄ ranging form 0.05 μg/kg to 50 μg/kg (n=4 subjects/group). Plasma samples were obtained at various time points before and after LTB₄ injections and the α-Defensin levels determined by ELISA. The data shown in FIG. 1 represent the mean ± standard error of α-Defensins (ng/mL plasma) of all subjects within each group. P values were determined by comparing the mean α-Defensin levels of each group that received LTB₄ versus the placebo group at the same time points. *p<0.001; **p<0.05. Plasma samples obtained before placebo or LTB₄ injections indicate that there were no significant differences in the basal levels of α-Defensins between the groups. In striking contrast to the placebo group, in vivo LTB₄ administration triggered dose-dependent release of α-Defensins in the plasma of subjects. The lowest dose of LTB₄ (0.05 pg/kg) administered did not significantly enhanced α-Defensins release. The second lowest dose of LTB₄ (0.5 μg/kg) administered, although not significant, caused a measurable increase in plasmatic α-Defensins, with maximal effect at two hours post-LTB₄ administration. The 5 μg/kg and 50 μg/kg doses of LTB₄ triggered a significant increase of α-Defensin release as soon as 30 minutes following LTB₄ injections. α-Defensin levels peaked (5-7 fold over the placebo group) at 2 hours (p<0.001) and remained above the control levels (p<0.05) for up to 6 hours post-LTB₄ administration. a-Defensins levels were back to the pre-injection levels when assayed 24 hours post-LTB₄ administration.

Considering that soluble anti-HIV factors include β-chemokines and that neutrophils are capable of producing MIP-1β, we measured the levels of this β-chemokine in plasma of individuals that received a placebo or LTB₄ (same cohorts as mentioned above). The data, expressed as the mean ± standard error of the plasmatic MIP-1 (pg/ml) levels, are presented in FIG. 2. Plasma samples from healthy subjects that received a placebo (n=3) or LTB₄ (5 and 50 μg/kg [n=4 subjects/group]) were obtained at various time points before and after LTB₄ injections. Data are expressed as mean ± absolute error of the mean of MIP-1β levels (pg/ml) for all subjects within each group. P values were determined by comparing the mean MIP-1β levels of each group that received LTB₄ versus the placebo group at the same time points. *p<0.025. Our data indicate that plasmatic MIP-1β levels determined before placebo or LTB₄ injections were identical. In contrast to the placebo group, individuals that received LTB₄ (5-50 μg/kg) showed an elevation in their plasmatic MIP-1β levels starting at 1 hour and reaching maximal levels at 2 hours post-LTB₄ injection. At 6 hours post-LTB₄ administration, the MIP-1β plasmatic levels had returned to original levels. Although the kinetics of MIP-1β production/accumulation were very similar for both groups, statistical significance was observed only for those that received the highest dose of LTB₄, a likely consequence of the small number of subjects (n=4) per group. Plasma samples from subjects that received the 2 lowest doses of LTB₄ were not tested for MIP-1β.

### EXAMPLE II

Sub-cutaneous Administration of LTB4 to Monkeys and Measurements of α-Defensin Release

Material and methods

Two macaques (Macaca fascicularis) were used for the study. For subcutaneous injections, the areas to be injected were first shaved one day before dosing. Volumes of 0.075 mL (monkey IJI) or 0.1 mL (monkey JKN) of LTB₄ solution were administered subcutaneously in the arm of the animal to compensate for slight difference between both monkeys. Venous blood samples were collected at various time points with all plasma isolation procedures performed at 4° C. Platelet-free plasma samples were stored at −80° C. until assayed for α-Defensins using a commercial ELISA kit (HyCult Biotechnology bv, The Netherlands).

Results

Effects of Subcutaneous LTB₄ Administration on Plasmatic α-Defensin Release in Monkeys

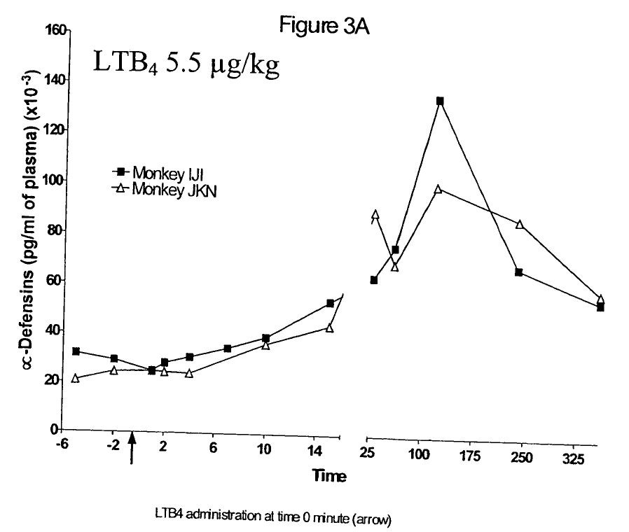

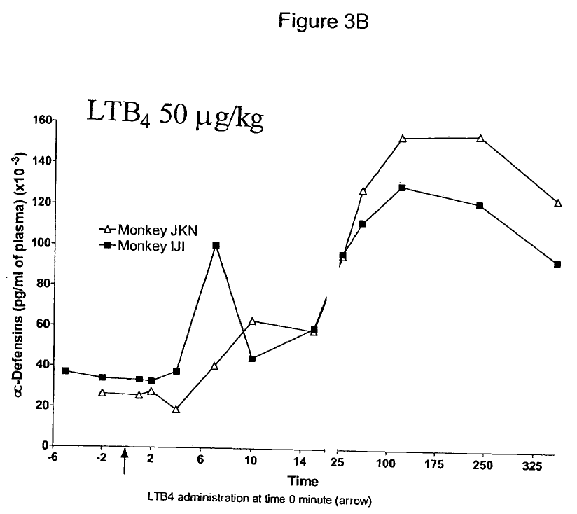

We next studied the change in plasmatic concentration of the antimicrobial peptides α-Defensins in monkeys following subcutaneous (s.c.) administration of LTB₄. Monkeys were injected s.c. with 5,5 μg/kg or 50 μg/kg of LTB₄ and blood samples were taken at various time points and used for (α-Defensins measurements in the plasma, using a commercial ELISA kit. The results obtained are presented in FIGS. 3A (5,5 μg/kg) and 3B (50 μg/kg). Two macaques (IJI and JKN) were injected subcutaneously with 5,5 μg/kg (A) or 50 μg/kg (B) of LTB₄. Blood samples were collected at various time points and α-Defensins levels measured in plasma samples using a commecial ELISA kit. Overall, the data obtained indicate that α-Defensins levels in plasma remain relatively stable up to 15 minutes post-LTB4 administration. α-Defensins levels started to increase by 30 minutes and peaked at two hours post LTB₄ administration. For monkeys that received 5,5 μg/kg of LTB₄, the α-Defensins levels started to drop by the fourth hour and continued to do so up to hour 6, time at which the experiment was ended. For monkeys that received 50 μg/kg, (α-Defensins levels remained at their highest up to 4 hours post injection, followed by a gradual decrease. In both sets (5,5 μg/kg and 50 μg/kg), the levels of α-Defensins remained above baseline values up to 6 hours following LTB₄ delivery

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for stimulating or increasing in vivo release of an α-defensin or MIP-1β in a human or animal comprising administering to a human or animal, a therapeutically effective amount of an exogenous LTB₄ agent.

2. The method according to claim 1, wherein said agent is leukotriene B₄[5S,12R-dihydroxy-6,8,10,14(Z,E,E,Z)-eicosatetraenoic acid].

3. The method according to claim 1, wherein said LTB₄ agent is selected from the group consisting of:
LTB₄, 14,15-dihydro-LTB₄ (“LTB₃”), 17,18-dehydro-LTB₄ (“LTB₅”), 19-hydroxy-LTB₄, 20-hydroxy-LTB₄, and 5(S)hydroperoxy, 5-keto, 5(R)-hydroxy, 5(R)-hydroperoxy, and 5-deoxy analogs thereof; leukotriene A₄ (“LTA₄”), 14,15-dihydro-LTA₄ (“LTA₃”), 17,18-dehydro-LTA₄ (“LTA₅”); 14,15-dihydro-LTA4 methyl ester, LTB₄ methyl ester;
12(R)-hydroxy-5,8,10,14(Z,Z,E,Z)-eicosatetraenoic acid (“12-HETE”), 5,6-dihydro-12-HETE, 14,15-dihydro-12-HETE, 17,18-dehydro-12-HETE and 12(R)-hydroperoxy, 12-keto, 12(S)-hydroxy and 12(S)-hydroperoxy analogs thereof; 5(S)-hydroxy-6,8,11,14(E,Z,Z,Z)-eicosatetraenoic acid (“5-HETE”), 14,15-dihydro-5-HETE, 17,18-dehydro-5-HETE, and 5(R)-hydroxy, 5-keto, 5(S)-hydroperoxy, 5(R)-hydroperoxy analogs thereof; 12-oxo-5,8,10(Z,Z,E)-dodecatrienoic acid, 15(S)-hydroxy-5,8,11,13(Z,Z,Z,E)-eicosatetraenoic acid (“15-HETE”), 5,6-dihydro-15-HETE, 17,18-dehydro-15-HETE and 15(R)-hydroxy, 15-keto, 15(S)-hydroperoxy, and 15(R)-hydroperoxy analogs thereof; 12(S)-hydroxy-5,8,10(Z,E,E)-heptadecatrienoic acid;
leukotrienes C₄, D₄ and E₄ and 14,15-dihydro or 17,18-dehydro analogs thereof; N-acyl or N-alkyl derivatives of leukotrienes C₄, D₄ and E₄, and 14,15-dihydro or 17,18-dehydro analogs thereof; 5,12-dihydroxy-6,8,10,14-eicosatetraenoic acid, and 14,15-dihydro or 17,18-dehydro analogs thereof; 5,6-dihydroxy-7,9,11,14-eicosatetraenoic acid, and 14,15-dihydro or 17,18-dehydro analogs thereof; 5,15-dihydroxy-6,8,11,13-eicosatetraenoic acid, and 17,18-dehydro analogs thereof; 8-hydroxy-11(12)-epoxy-5,9,14-eicosatrienoic acid, hepoxilin A₃, and 5,6-dihydro or 14,15-dihydro or 17,18-dehydro analogs thereof; 10-hydroxy-11(12)-epoxy-5,8,14-eicosatrienoic acid, hepoxilin B₃, and 5,6-dihydro or 14,15-dihydro or 17,18-dehydro analogs thereof; 8,11,12-trihydroxy-5,9,14-eicosatrienoic acid, trioxilin A₃, and 5,6-dihydro or 14,15-dihydro or 17,18-dehydro analogs thereof; 10,11,12-trihydroxy-5,8,14-eicosatrienoic acid, trioxilin B₃, and 5,6-dihydro or 14,15-dihydro or 17,18-dehydro analogs thereof; 5(S),15(S)-dihydroxy-6,8,11,13(E,Z,Z,E)-eicosatetraenoic acid; 11(12)-epoxy-5,7,9,14-eicosatetraenoic acid, and 14,15-dihydro or 17,18-dehydro analogs thereof; 11,12-dihydroxy-5,7,9,14-eicosatetraenoic acid, and 14,15-dihydro or 17,18-dehydro analogs thereof; 8(9)-epoxy-5,10,12,14-eicosatetraenoic acid, and 5,6-dihydro or 17,18-dehydro analogs thereof; 8,9-dihydroxy-5,10,12,14-eicosatetraenoic acid, and 5,6-dihydro or 17,18-dehydro analogs thereof; 8,15-dihydroxy-5,9,11,13-eicosatetraenoic acid, and 5,6-dihydro or 17,18-dehydro analogs thereof; 14(15)-epoxy-5,8,10,12-eicosatetraenoic acid, and 5,6-dihydro or 17,18-dehydro analogs thereof; 14,15-dihydroxy-5,8,10,12-eicosatetraenoic acid, and 5,6-dihydro or 17,18-dehydro analogs thereof; 5-hydroxy-14(15)-epoxy-6,8,10,12-eicosatetraenoic acid, and 17,18-dehydro analogs thereof; 5,14,15-trihydroxy-6,8,10,12-eicosatetraenoic acid, lipoxin B₄, and 17,18-dehydro analogs thereof;
5,6,15-trihydroxy-7,9,11,13-eicosatetraenoic acid, lipoxin A₄, and 17,18-dehydro analogs thereof; 5(6)-epoxy-15-hydroxy-7,9,11,13-eicosatetraenoic acid, and 17,18-dehydro analogs thereof; 5-hydroxy-6,8,11,14-eicosatetraenoic acid, and 14,15-dihydro or 17,18-dehydro analogs thereof; 9-hydroxy-5,7,11,14-eicosatetraenoic acid, and 14,15-dihydro or 17,18-dehydro analogs thereof; 11-hydroxy-5,8,12,14-eicosatetraenoic acid, and 5,6-dihydro or 17,18-dehydro analogs thereof; 12-hydroxy-5,8,10,14-eicosatetraenoic acid, and 5,6-dihydro or 14,15-dihydro or 17,18-dehydro analogs thereof; 15-hydroxy-5,8,11,13-eicosatetraenoic acid, and 5,6-dihydro or 17,18-dehydro analogs thereof; 9-hydroxy-10,12-octadecadienoic acid and isomers thereof; 13-hydroxy-9,11-octadecadienoic acid and isomers thereof; 12(R)-hydroxy-5,8,14(Z,Z,Z)-eicosatrienoic acid and isomers thereof; 5(6)oxido- or 5,6-dihydroxy-8,11,14-eicosatrienoic acid, and 14,15-dihydro or 17,18-dehydro analogs thereof; 8(9)-oxido- or 8,9-dihydroxy-5,11,14-eicosatrienoic acid, and 5,6-dihydro or 14,15-dihydro or 17,18-dehydro analogs thereof; 11(12)-oxido- or 11,12-dihydroxy-5,8,14-eicosatrienoic acid, and 5,6-dihydro or 14,15-dihydro or 17,18-dehydro analogs thereof; 14(15)-oxido- or 14,15-dihydroxy-5,8,11-eicosatrienoic acid, and 5,6-dihydro or 17,18-dehydro analogs thereof; 8-hydroxy-5,9,11,14-eicosatetraenoic acid, isomers thereof and 5,6-dihydro or 14,15-dihydro or 17,18-dehydro analogs thereof;
20,20,20-trifluoromethyl-LTB₄; 19-methyl-LTB₄, 19,19-dimethyl-LTB₄, 19-fluoro-LTB₄, 19,19-difluoro-LTB_{4, 18,20}-difluro-LTB₄, 20-fluoro-LTB₄; 3-thio-LTB₄, 3-hydroxy-LTB_{4;}
3-methyl-LTB₄, 3,3-dimethyl-LTB₄, 3-fluoro-LTB₄, 3,3-difluoro-LTB₄, 2,3-difluoro-LTB₄;
LTB₄ methylsulfonylamide, LTB₄ methylamide, and 1-tetrazole LTB₄.

4. The method according to claim 1, wherein said LTB₄ agent is a salt thereof.

5. The method according to claim 1, wherein said LTB₄ agent is an ester derivative thereof.

6. The method according to claim 1, wherein said LTB₄ agent is an ether derivative thereof.

7. The method of claim 1, wherein said method is for the treatment or prophylaxis of a microbial infection in humans and animals.

8. The method according to claim 7, wherein said method is for the treatment or prophylaxis of HIV.

9. The method according to claim 7, wherein said method is for the treatment or prophylaxis of anthrax.

10. Use of an LTB₄ agent as defined in claim 3 for the preparation of a medicament for the treatment or prophylaxis of a microbial infection in humans and animals.

11. The use according to claim 10, wherein said microbial infection is HIV.

12. The use according to claim 10, wherein said microbial infection is anthrax.

13. The method of claim 1, wherein said administration is effected orally, intraarterially, intravenously, intraperitoneally, subcutaneously, intramuscularly, sublingually, intranasally, parenterally, topically, by inhalation or by suppository.

14. A method of treating a microbial infection in humans or animals, comprising administering an LTB₄ agent of claim 3.

15. The method of claim 14, wherein the microbial infection is HIV.

16. The method of claim 14, wherein the microbial infection is anthrax.
